(19) United States
(12) Patent Application Publication
(10) Pub. No.: US 2024/0316079 A1
(43) Pub. Date: Sep. 26, 2024

(54) USE OF AKR1C3-ACTIVATED COMPOUND

(71) Applicant: OBI PHARMA, INC., Taipei City (CN)

(72) Inventors: Jianxin Duan, Shenzhen (CN); Fanying Meng, San Francisco, CA (US); Tianyang Qi, Shenzhen (CN); Chun-Chung Wang, Taipei City (CN); Lu-Tzu Chen, Taipei City (CN); Wan-Fen Li, Taipei City (CN); Ming-Tain Lai, Taipei City (CN)

(21) Appl. No.: 18/261,137

(22) PCT Filed: Feb. 26, 2021
(86) PCT No.: PCT/CN2021/078115

§ 371 Date: Jul. 12, 2023

(51) Int. Cl. A61K 31/675; A61K 31/404; A61K 31/513; A61K 31/573; A61K 31/58; A61K 31/7068; A61P 35/00

(52) U.S. Cl. CPC A61K 31/675; A61K 31/404; A61K 31/513; A61K 31/573; A61K 31/58; A61K 31/7068; A61P 35/00

(57) ABSTRACT

Provided are the use of the compound 1-(3-(3-N,N-dimethylaminocarbonyl)phenoxyl-4-mtrophenyl)-1-ethyl-N,N′-bis(ethylene)phosphoramidate, or a pharmaceutically acceptable salt, isotopic variant or solvate thereof in the manufacture of a medicament for treating cancer, and a composition which comprises the above compound and at least one anti-cancer drug.

### TECHNICAL FIELD

The present invention relates to medical use of the compound 1-(3-(3-N,N-dimethylaminocarbonyl)phenoxyl-4-mtrophenyl)-1-ethyl-N,N′-bis(ethylene)phosphoramidate, or a pharmaceutically acceptable salt, isotopic variant or solvate thereof, and to a composition which comprises the above compound and at least one anti-cancer drug and its medical use.

### BACKGROUND ART

Cancer is one of the major causes of human morbidity and mortality. Cancer treatment is challenging because it is difficult to kill cancer cells without damaging or killing normal cells. Damaging or killing normal cells during cancer treatment is a cause of adverse side effects in patients and can limit the amount of anti-cancer drug administered to a cancer patient.

Aldo-keto reductase 1C3 (AKR1C3) is also known as type 5, 17β-hydroxysteroid dehydrogenase (17β-HSD) and prostaglandin F synthase. AKR1C3 is one member of the 15 gene families of aldo-keto reductases (AKRs). AKR1C3 was originally cloned from human prostate⁽¹⁾ and placenta⁽²⁾ cDNA libraries. AKR1C3 is a monomeric, cytosolic, NAD(P) (H)-dependent oxidoreductase with 323 amino acids and a molecular weight of 37 kDa⁽¹⁾. AKR1C3 shares high sequence homology with the related human AKR1C family, including AKR1C1, AKR1C2, and AKR1C4. AKR1C3 catalyzes androgen, estrogen, progesterone, and prostaglandin (PG) metabolism and is subsequently involved in the regulation of nuclear receptor activities^{(3,4)}. AKR1C3 is expressed in normal tissues including steroid hormone-dependent and steroid hormone-independent cells with an average low expression level except in liver, kidney, and small intestine⁽⁵⁾. Many studies have demonstrated that AKR1C3 is abnormally overexpressed in many malignant solid and hematologic tumors. The data show that more than 50% of hepatoma, bladder, renal, and gastric cancers were detected with high expression of AKR1C3 with immunohistochemistry scores (IHC score) ≥4 on a scale of 0 to 6⁽⁶⁾. AKR1C3 is highly expressed in non-small cell lung cancer (NSCLC) but not in small cell-lung cancer⁽⁷⁾.

AKR1C3 upregulation in cancer is reported to be associated with metastasis of castrate-resistant prostate cancer (CRPC⁽⁸⁾) and colorectal cancer (CRC⁽⁹⁾), and is also linked to poor prognosis and a low survival rate (10,11) In addition, many types of treatment resistance are attributed to the overexpression of AKR1C3. It has been reported that chemotherapy resistance to doxorubicin^{(12,13)} enzalutamide⁽¹⁴⁾, abiraterone⁽¹⁵⁾ and methotrexate⁽¹⁶⁾ is directly related to high AKR1C3 expression in cells. Radiotherapy resistance in esophageal cancer⁽¹⁷⁾, prostate cancer⁽¹⁸⁾ and NSCL cancer cells⁽¹⁹⁾ is associated with AKR1C3 overexpression. The main mechanism of action of AKR1C3 against ionizing radiation is to reduce ROS (reactive oxygen species) in cells, to increase PGF2α which subsequently leads to MAP kinase activation and PPARγ inhibition resulting in a significant reduction in DNA damage⁽¹⁸⁾. Immunotherapy resistance is also attributed to AKR1C3 high expression. One study has shown that high expression of AKR1C3 is associated with the failure of PD-1-targeted therapies in PD-L1 positive patients with advanced renal cell carcinoma (RCC) based on whole genome microarray and multiplex quantitative (q)RT-PCR gene expression analysis⁽²⁰⁾. Due to tumor-specific overexpression of AKR1C3, the design of AKR1C3-activated prodrugs becomes an attractive approach to specifically target cancer. One such example is the AKR1C3-activated prodrug, PR104, which exhibited good anti-tumor activity in vitro and in vivo^{(6,21)} although it was originally designed as a hypoxia-activated prodrug⁽²²⁻²⁴⁾.

Anti-cancer prodrug of the present application of Formula I-1 (denoted by 3424 herein) is a chemically synthesized potent nitrogen mustard, which is selectively cleaved to the cytotoxic aziridine (denoted by 2660 herein) by AKR1C3 in the presence of NADPH. The active molecule 2660 released by 3424 is similar to the standard chemotherapeutic drugs thiotepa and mitomycin C, which leads to alkylation and cross-linking of DNA at the N7 (or 06) position of guanine. Prodrug 3424 is currently under development by Ascentawits Pharmaceuticals, LTD in Asian countries and by OBI Pharma, Inc. in countries outside Asia (drug code OBI-3424) for the treatment of malignant tumors. Prodrug 3424 is currently being investigated in multiple Phase I clinical trials in the US (NCT04315324 & NCT03592264) and in China (CXHL1900137 & CXHL2000263) to treat more than 14 types of human cancer, including solid tumors and hematologic malignancies. Due to the high expression of AKR1C3 in tumors, prodrug 3424 is designed to be specifically activated in tumors but spared in normal cells which express low levels of AKR1C3 to achieve tumor-specific targeting. Furthermore, tumor-selective activation of 3424 is distinguishable from non-selective traditional alkylating agents, such as cyclophosphamide and ifosfamide, indicating that 3424 has the potential to become a broad-spectrum, highly selective anti-tumor drug. Prodrug 3424 was reported to exhibit potent efficacy against preclinical models of T-ALL in vitro and in vivo^{(25,26)}.

In the presence of NADPH, reduction of 3424 is mediated by AKR1C3 to release the cytotoxic moiety 2660, which is an aziridine bis-alkylating agent, leading to cross-linking of DNA at the N7 (or 06) position of guanine, and subsequent cell death.

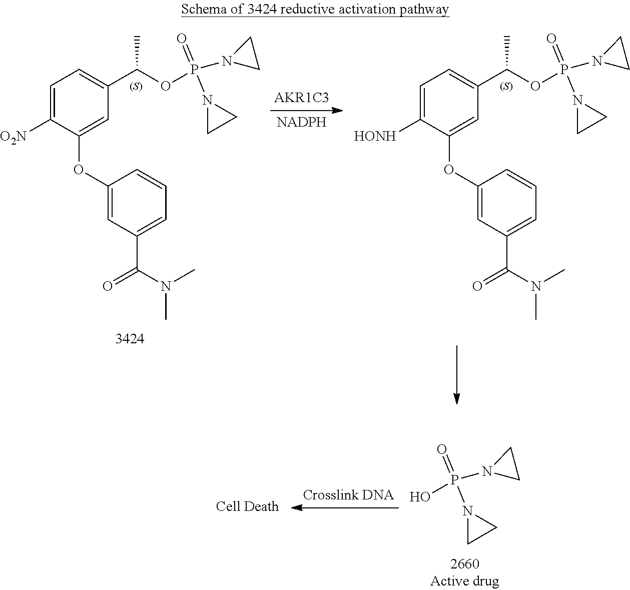

Prodrugs designed to target cancer cells have emerged as an attractive strategy for cancer therapy in recent years; however, many prodrugs failed in Phase 3 clinical trials due to a lack of valid biomarkers to select patients⁽³³⁾. Given that the AKR1C3 expression can be assessed using RT-PCR or immunohistochemistry, 3424 can be developed in a clinically efficient manner by selecting patients who have high AKR1C3 expression and are most likely to respond to the prodrug. AKR1C3 has been demonstrated to be overexpressed upon acquisition of chemoresistance^{(13,14)}, radioresistance⁽¹⁹⁾ and immunoresistance⁽²⁰⁾. In addition, cancers with homologous recombination deficiency (HRD) such as ovarian, breast, and pancreatic cancers, are known to be sensitive to DNA damaging agents⁽³⁴⁾. As a DNA alkylator, 3424 may also be a good candidate drug to treat HRD cancers that have AKR1C3 expression.

There remains a need for a compound suitable for treating cancer patients, which is a selective AKR1C3 reductase activated prodrug, and a novel, selective and broad anti-cancer agent. The present invention meets this need.

### SUMMARY OF THE INVENTION

The present invention, based on the compounds or pharmaceutically acceptable salts, or solvates thereof as disclosed in Patent Application No. PCT/US2016/021581 (WO2016/145092) and Patent Application No. PCT/US2016/062114 (WO2017/087428), provides medical use of the compounds, and provides compositions comprising the compounds or pharmaceutically acceptable salts, isotopic variants or solvates thereof and their anti-cancer medical use.

In one aspect, the present invention provides use of the compound 1-(3-(3-N,N-dimethylaminocarbonyl)phenoxyl-4-mtrophenyl)-1-ethyl-N,N′-bis(ethylene)phosphoramidate having the following Formula I, or a pharmaceutically acceptable salt, isotopic variant or solvate thereof in the manufacture of a medicament for treating cancer in a patient

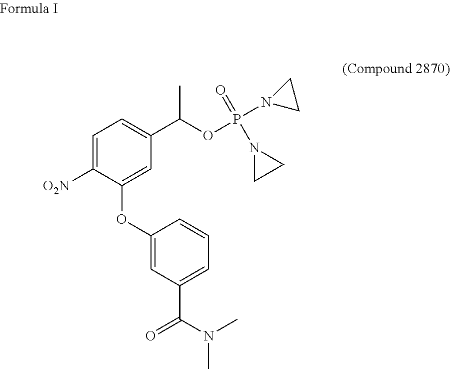 [SMILES: CC(OP(=O)(N1CC1)N1CC1)c1ccc([N+](=O)[O-])c(Oc2cccc(C(=O)N(C)C)c2)c1]

wherein the AKR1C3 reductase level of the cancer is represented by the AKR1C3 protein level or RNA level and is equal to or greater than a predetermined value. AKR1C3 levels are measured following routine methods well known to the skilled artisan.

According to particular embodiments of the invention, the compound is
(S)-1-(3-(3-N,N-dimethylaminocarbonyl)phenoxyl-4-mtrophenyl)-1-ethyl-N,N′-bis(ethylene)phosphoramidate having the following Formula I-1, or
(R)-1-(3-(3-N,N-dimethylaminocarbonyl)phenoxyl-4-mtrophenyl)-1-ethyl-N,N′-bis(ethylene)phosphoramidate having the following Formula I-2 (denoted by 3423 herein)

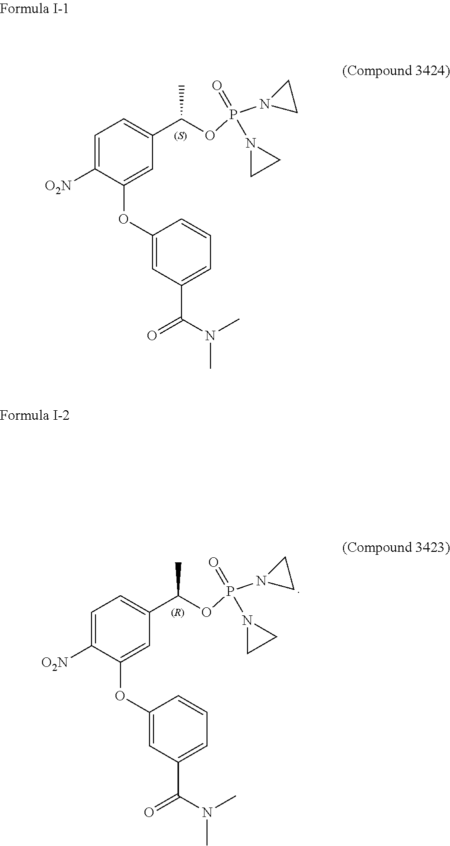

The preparation of the compound of Formula I, Formula I-1 or Formula I-2 is disclosed in PCT Application No. PCT/US2016/021581 (WO2016/145092) and Patent Application No. PCT/US2016/062114 (WO2017/087428), the disclosures of which are incorporated herein by reference in its entirety. Herein, compound 2870 is a racemic mixture of R-enantiomer 3423 and S-enantiomer 3424 at 1:1 ratio.

Herein, the salts may be basic salts, including the salts of the compounds with an inorganic base (such as alkali metal hydroxide and alkaline earth metal hydroxide) or with an organic base (such as monoethanolamine, diethanolamine or triethanolamine). Alternatively, the salts may be acid salts, including the salts of the compounds with an inorganic acid (such as hydrochloric acid, hydrobromic acid, hydroiodic acid, nitric acid, perchloric acid, sulfuric acid or phosphoric acid) or with an organic acid (such as methanesulfonic acid, trifluoromethanesulfonic acid, ethanesulfonic acid, benzenesulfonic acid, p-toluenesulfonic acid, fumaric acid, oxalic acid, maleic acid and citric acid). It is a well-known technology in the art to select and prepare acceptable salts, solvates, and the like of a compound.

According to particular embodiments of the invention, the compound of Formula I-1 or Formula I-2 has an enantiomeric excess of no less than 80%. Preferably, the compound has an enantiomeric excess of no less than 90%, more preferably, no less than 95%.

According to particular embodiments of the invention, the compound of Formula I-1 or Formula I-2 is substantially pure.

According to particular embodiments of the invention, the cancer is liver cancer, hepatocellular carcinoma (HCC), lung cancer, melanoma, prostate cancer, breast cancer, leukemia, esophageal cancer, renal cancer, gastric cancer, colon cancer, brain cancer, bladder cancer, cervical cancer, ovarian cancer, head and neck cancer, endometrial cancer, pancreatic cancer, a sarcoma cancer, or rectal cancer.

According to particular embodiments of the invention, the cancer is liver cancer, non-small cell lung cancer, castrate-resistant prostate cancer, gastric cancer, renal cell carcinoma or pancreatic cancer.

The dosage of the medicament used for treating cancer, or the dosage of the compound or salt, isotopic variant or solvate thereof, or the other anti-cancer drug contained in the medicament usually depends on the specific compound applied, the patient, the specific disease or condition and the severity thereof, the route and frequency of administration and the like, and needs to be determined by the attending physician according to specific conditions. For example, when the composition or medicament provided by the present invention is administered by the oral route, the dosage may be 0.1 to 30 mg/7 days, preferably 1 to 10 mg/7 days, more preferably 5 mg/day; the dosage may be administered once every 7 days or divided into two dosages for administration twice every 7 days; preferably, the dosage is administered once every 7 days.

The medicament can be any dosage form for clinical administration, such as tablets, suppositories, dispersible tablets, enteric-coated tablets, chewable tablets, orally disintegrating tablets, capsules, sugar coated agents, granules, dry powders, oral solutions, a small needle for injection, lyophilized powder for injection, or infusion solutions.

In another aspect, the invention provides a method for treating cancer in a patient in need thereof, comprising the step of administering to the patient an effective amount of the compound 1-(3-(3-N,N-dimethylaminocarbonyl)phenoxyl-4-mtrophenyl)-1-ethyl-N,N′-bis(ethylene)phosphoramidate having the following Formula I, or a pharmaceutically acceptable salt, isotopic variant or solvate thereof.

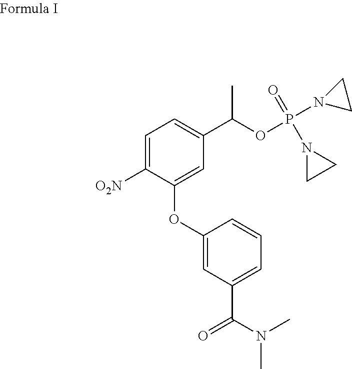 [SMILES: CC(OP(=O)(N1CC1)N1CC1)c1ccc([N+](=O)[O-])c(Oc2cccc(C(=O)N(C)C)c2)c1]

wherein the AKR1C3 reductase level of the cancer is represented by the AKR1C3 protein level or RNA level and is equal to or greater than a predetermined value.

According to particular embodiments of the invention, the compound is
(S)-1-(3-(3-N,N-dimethylaminocarbonyl)phenoxyl-4-mtrophenyl)-1-ethyl-N,N′-bis(ethylene)phosphoramidate having the following Formula I-1, or
(R)-1-(3-(3-N,N-dimethylaminocarbonyl)phenoxyl-4-mtrophenyl)-1-ethyl-N,N′-bis(ethylene)phosphoramidate having the following Formula I-2

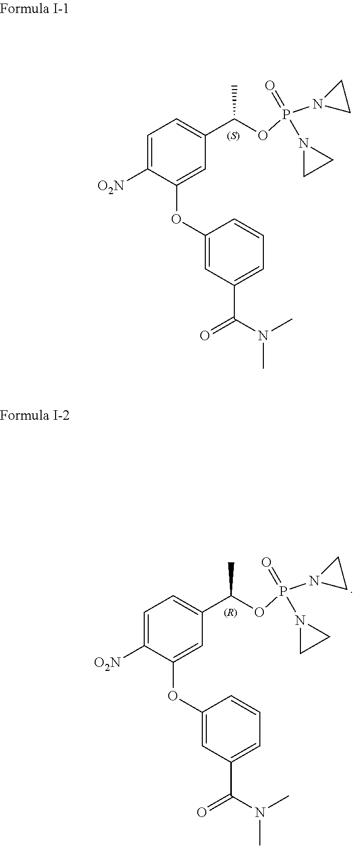

According to particular embodiments of the invention, the cancer is liver cancer, hepatocellular carcinoma (HCC), lung cancer, melanoma, prostate cancer, breast cancer, leukemia, esophageal cancer, renal cancer, gastric cancer, colon cancer, brain cancer, bladder cancer, cervical cancer, ovarian cancer, head and neck cancer, endometrial cancer, pancreatic cancer, a sarcoma cancer, or rectal cancer.

According to particular embodiments of the invention, the cancer is liver cancer, non-small cell lung cancer, castrate-resistant prostate cancer, gastric cancer, renal cell carcinoma or pancreatic cancer.

According to particular embodiments of the invention, the method further comprises a step for measuring the content of AKR1C3 reductase of cancer cells in a patient using AKR1C3 antibodies, where the content of AKR1C3 reductase is measured to be equal to or greater than the predetermined value, the compound is administered to the patient.

In another aspect, the invention provides a method for inhibiting the growth of a cell, comprising the step of contacting the cell with an effective amount of compound 1-(3-(3-N,N-dimethylaminocarbonyl)phenoxyl-4-mtrophenyl)-1-ethyl-N,N′-bis(ethylene)phosphoramidate having Formula I, or a pharmaceutically acceptable salt, isotopic variant or solvate thereof; wherein the AKR1C3 reductase level of the cell is represented by the AKR1C3 protein level or RNA level and is equal to or greater than a predetermined value.

According to particular embodiments of the invention, the cell is a cancerous cell.

According to particular embodiments of the invention, the method further comprises a step for measuring the content of AKR1C3 reductase of cell using AKR1C3 antibodies, where the content of AKR1C3 reductase is measured to be equal to or greater than the predetermined value, the compound is contacted with the cell.

In another aspect, the invention provides use of the compound 1-(3-(3-N,N-dimethylaminocarbonyl)phenoxyl-4-mtrophenyl)-1-ethyl-N,N′-bis(ethylene)phosphoramidate having Formula I, or a pharmaceutically acceptable salt, isotopic variant or solvate thereof in the manufacture of a medicament for inhibiting the growth of a cell; wherein the AKR1C3 reductase level of the cell is represented by the AKR1C3 protein level or RNA level and is equal to or greater than a predetermined value.

According to particular embodiments of the invention, the cell is a cancerous cell.

In another aspect, the invention provides a composition, which comprising: 1) the compound 1-(3-(3-N,N-dimethylaminocarbonyl)phenoxyl-4-mtrophenyl)-1-ethyl-N,N′-bis(ethylene)phosphoramidate having Formula I, or a pharmaceutically acceptable salt, isotopic variant or solvate thereof; and 2) at least one other anti-cancer drug.

According to particular embodiments of the invention, the compound is
(S)-1-(3-(3-N,N-dimethylaminocarbonyl)phenoxyl-4-mtrophenyl)-1-ethyl-N,N′-bis(ethylene)phosphoramidate having Formula I-1, or
(R)-1-(3-(3-N,N-dimethylaminocarbonyl)phenoxyl-4-mtrophenyl)-1-ethyl-N,N′-bis(ethylene)phosphoramidate having Formula I-2.

According to particular embodiments of the invention, the anti-cancer drug is selected from the group consisting of gemcitabine, 5-flurouracie (5-FU), sunitinib, abiraterone acetate, prednisolone, erlotinib, meturedepa, uredepa, altretamine, imatinib, triethylenemelamine, trimethylmelamine, chlorambucil, chlornaphazine, estramustine, gefitinib, mechlorethamine, mechlorethamine oxide hydrochloride, melphalan, novembichin, phenesterine, prednimustine, trofosfamide, uracil mustard, carmustine, chlorozotocin, fotemustine, nimustine, ranimustine, dacarbazine, mannomustine, mitobronitol, mitolactol, pipobroman, aclacinomycins, actinomycin, anthramycin, azaserine, bleomycin, cactinomycin, carubicin, carzinophilin, chromomycin, dactinomycin, daunorubicin, daunomycin, 6-diazo-5-oxo-1-norleucine, mycophenolic acid, nogalamycin, olivomycin, peplomycin, plicamycin, porfiromycin, puromycin, streptonigrin, streptozocin, tubercidin, ubenimex, zinostatin, zorubicin, denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, tegafur, L-asparaginase, pulmozyme, aceglatone, aldophosphamide glycoside, aminolevulinic acid, amsacrine, bestrabucil, bisantrene, defofamide, demecolcine, diaziquone, elfornithine, elliptinium acetate, etoglucid, flutamide, hydroxyurea, interferon-alpha, interferon-beta, interferon-gamma, interleukin-2, lentinan, mitoguazone, mitoxantrone, mopidamol, nitracrine, pentostatin, phenamet, pirarubicin, podophyllinic acid, 2-ethylhydrazide, procarbazine, razoxane, sizofiran, spirogermanium, paclitaxel, tamoxifen, erlotonib, teniposide, tenuazonic acid, triaziquone, 2,2′,2″-trichlorotriethylamine, urethan, vinblastine, and vincristine.

According to particular embodiments of the invention, the anti-cancer drug is selected from the group consisting of gemcitabine, abiraterone acetate, prednisolone, 5-FU, sunitinib, or the combination of abiraterone acetate and prednisolone.

According to particular embodiments of the invention, in the case where the cancer is renal cell carcinoma (RCC), the anti-cancer drug is selected from the group consisting of gemcitabine and sunitinib; in the case where the cancer is gastric cancer, the anti-cancer drug is 5-FU; in the case where the cancer is castrate-resistant prostate cancer (CRPC), the anti-cancer drug is selected from the group consisting of abiraterone acetate and prednisolone or their combination.

According to particular embodiments of the invention, the composition further comprises a pharmaceutically acceptable excipient. Preferably, the excipient is selected from inert diluents, dispersing and/or granulating agents, surface active agents and/or emulsifiers, disintegrating agents, binding agents, preservatives, buffering agents, lubricating agents and oils.

In another aspect, the invention provides use of a composition according to the invention in the manufacture of a medicament for treating cancer in a patient.

According to particular embodiments of the invention, the cancer is liver cancer, hepatocellular carcinoma (HCC), lung cancer, melanoma, prostate cancer, breast cancer, leukemia, esophageal cancer, renal cancer, gastric cancer, colon cancer, brain cancer, bladder cancer, cervical cancer, ovarian cancer, head and neck cancer, endometrial cancer, pancreatic cancer, a sarcoma cancer, or rectal cancer.

According to particular embodiments of the invention, the AKR1C3 reductase level of the cancer is equal to or greater than a predetermined value.

According to particular embodiments of the invention, the cancer is liver cancer, non-small cell lung cancer, castrate-resistant prostate cancer, gastric cancer, renal cell carcinoma or pancreatic cancer.

In another aspect, the invention provides a method for treating cancer in a patient in need thereof, comprising the step of administering to the patient an effective amount of the composition according to the invention.

According to particular embodiments of the invention, the cancer is liver cancer, hepatocellular carcinoma (HCC), lung cancer, melanoma, prostate cancer, breast cancer, leukemia, esophageal cancer, renal cancer, gastric cancer, colon cancer, brain cancer, bladder cancer, cervical cancer, ovarian cancer, head and neck cancer, endometrial cancer, pancreatic cancer, a sarcoma cancer, or rectal cancer.

According to particular embodiments of the invention, the AKR1C3 reductase level of the cancer is equal to or greater than a predetermined value.

According to particular embodiments of the invention, the cancer is liver cancer, non-small cell lung cancer, castrate-resistant prostate cancer, gastric cancer, renal cell carcinoma or pancreatic cancer.

According to particular embodiments of the invention, the method further comprises a step for measuring the content of AKR1C3 reductase of cancer cells in a patient using AKR1C3 antibodies, where the content of AKR1C3 reductase is measured to be equal to or greater than the predetermined value, the composition is administered to the patient.

### BRIEF DESCRIPTION OF DRAWINGS

FIG. 1 depicts AKR1C3-dependent 3424 activation. (A) 3424 reduction; (B) 2660 production.

FIG. 2 depicts the detection of AKR1C3 protein expression in liver cancer cells by Western blot.

FIG. 3 depicts AKR1C3-dependent in vitro cytotoxicity of 3424. (A) Correlation between AKR1C3 protein expression and 3424 IC₅₀ in liver cancer cells (left); Correlation between AKR1C3 RNA expression and 3424 IC₅₀ in liver cancer cells (middle); Correlation between AKR1C3 RNA expression with 3424 IC₅₀ in NSCLC cancer cells (right); (B) AKR1C3-specific inhibitor, 3021 efficiently inhibited cytotoxicity of 3424 (left), 3423 (middle) and racemic mixture 2870; (C) Compound 2870 induced concentration-dependent DNA cross-linking.

FIG. 4 depicts anti-tumor activity of 3424 in various human cell line derived xenograft (CDX) models. HepG2 liver orthotopic model (A and B). VCap castration-resistance prostate cancer in castrated male BALB/c nude mice (C), SNU-16 gastric cancer in female BALB/c nude mice (D) and A498 renal cell carcinoma in female SCID mice (E and F). Animals were treated with various concentrations of 3424, standard of care therapies, and the combination as indicated in the legend; and wherein, “AA” represents for Abiraterone Acetate, “P” represents for Prednisolone, “S” represents for Sunitinib, and “G” represents for Gemzar.

FIG. 5 depicts anti-tumor activity of 3424 in subcutaneous lung cancer model H460 CDX model.

FIG. 6 depicts the measurement of serum prostate specific antigen (PSA) after treatment at the time indicated.

FIG. 7 depicts anti-tumor activity of 3424 against a panel of PDXs. Pancreatic cancer PA1280 (A), gastric cancer GA6201(B), and Lung cancer LU2505 with higher AKR1C3 expression (C) and lung cancer LU2057 with low AKR1C3 expression (D)

### DETAILED DESCRIPTION OF THE INVENTION

The present invention will be described below with reference to specific examples. Those skilled in the art could understand that these examples are only used for describing the invention and do not in any way limit its scope.

### Definitions

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

All numerical designations, e.g., pH, temperature, time, concentration, and weight, including ranges of each thereof, are approximations that typically may be varied (+) or (−) by increments of 0.1, 1.0, or 10.0, as appropriate. All numerical designations may be understood as preceded by the term “about”. Reagents described herein are exemplary and equivalents of such may be known in the art.

“A”, “an” and “the” include plural referents unless the context clearly dictates otherwise. Thus, for example, reference to a compound refers to one or more compounds or at least one compound. As such, the terms “a” (or “an”), “one or more”, and “at least one” are used interchangeably herein.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

“Administering” or “administration of” a drug to a patient (and grammatical equivalents of this phrase) refers to direct administration, which may be administration to a patient by a medical professional or may be self-administration, and/or indirect administration, which may be the act of prescribing a drug. For example, a physician who instructs a patient to self-administer a drug and/or provides a patient with a prescription for a drug is administering the drug to the patient.

“Cancer” refers to leukemias, lymphomas, carcinomas, and other malignant tumors, including solid tumors, of potentially unlimited growth that can expand locally by invasion and systemically by metastasis. Examples of cancers include, but are not limited to, cancer of the adrenal gland, bone, brain, breast, bronchi, colon and/or rectum, gallbladder, head and neck, kidney, larynx, liver, lung, neural tissue, pancreas, prostate, parathyroid, skin, stomach, and thyroid. Certain other examples of cancers include, acute and chronic lymphocytic and granulocytic tumors, adenocarcinoma, adenoma, basal cell carcinoma, cervical dysplasia and in situ carcinoma, Ewing's sarcoma, epidermoid carcinomas, giant cell tumor, glioblastoma multiforma, hairy-cell tumor, intestinal ganglioneuroma, hyperplastic corneal nerve tumor, islet cell carcinoma, Kaposi's sarcoma, leiomyoma, leukemias, lymphomas, malignant carcinoid, malignant melanomas, malignant hypercalcemia, marfanoid habitus tumor, medullary carcinoma, metastatic skin carcinoma, mucosal neuroma, myeloma, mycosis fungoides, neuroblastoma, osteo sarcoma, osteogenic and other sarcoma, ovarian tumor, pheochromocytoma, polycythermia vera, primary brain tumor, small-cell lung tumor, squamous cell carcinoma of both ulcerating and papillary type, hyperplasia, seminoma, soft tissue sarcoma, retinoblastoma, rhabdomyosarcoma, renal cell tumor, topical skin lesion, veticulum cell sarcoma, and Wilm's tumor.

The term “contacting” or “contact” is meant to refer to bringing together of a therapeutic agent and cell or tissue such that a physiological and/or chemical effect takes place as a result of such contact. Contacting can take place in vitro, ex vivo, or in vivo. In one embodiment, a therapeutic agent is contacted with a cell in cell culture (in vitro) to determine the effect of the therapeutic agent on the cell. In another embodiment, the contacting of a therapeutic agent with a cell or tissue includes the administration of a therapeutic agent to a subject having the cell or tissue to be contacted.

The terms “optically active” refers to a collection of molecules, which has an enantiomeric excess of no less than about 10%, no less than about 20%, no less than about 30%, no less than about 40%, no less than about 50%, no less than about 60%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, no less than about 99.8%, or no less than about 99.9%. In certain embodiments, the enantiomeric excess for an optically active compound is no less than about 90%, no less than about 95%, no less than about 98%, or no less than about 99%. An enantiomeric excess of a compound can be determined by any standard methods used by one of ordinary skill in the art, including, but not limited to, chiroptical chromatography (gas chromatography, high-performance liquid chromatography, and thin-layer chromatography) using an optically active stationary phase, isotopic dilution, electrophoresis, calorimetry, polarimetry, NMR resolution methods with chiral derivatization, and NMR methods with a chiral solvating agent or chiral shift reagent.

In describing an optically active compound, the prefixes R and S are used to denote the absolute configuration of the molecule about its chiral center(s).

The terms “substantially pure” means sufficiently homogeneous to appear free of readily detectable impurities as determined by standard analytical methods used by one of ordinary skill in the art, including, but not limited to, thin layer chromatography (TLC), gel electrophoresis, high performance liquid chromatography (HPLC), gas chromatography (GC), nuclear magnetic resonance (NMR), and mass spectrometry (MS); or sufficiently pure such that further purification would not detectably alter the physical, chemical, biological, and/or pharmacological properties, such as enzymatic and biological activities, of the substance. In certain embodiments, “substantially pure” refers to a collection of molecules, wherein at least about 50%, at least about 70%, at least about 80%, at least about 90%, at least about 95%, at least about 98%, at least about 99%, or at least about 99.5% by weight of the molecules are a single stereoisomer of a compound, as determined by standard analytical methods.

“Patient” and “subject” are used interchangeably to refer to a mammal in need of treatment for cancer. Generally, the patient is a human. Generally, the patient is a human diagnosed with cancer. In certain embodiments, a “patient” or “subject” may refer to a non-human mammal used in screening, characterizing, and evaluating drugs and therapies, such as, a non-human primate, a dog, cat, rabbit, pig, mouse or a rat.

“Prodrug” refers to a compound that, after administration, is metabolized or otherwise converted to a biologically active or more active compound (or drug) with respect to at least one property. A prodrug, relative to the drug, is modified chemically in a manner that renders it, relative to the drug, less active or inactive, but the chemical modification is such that the corresponding drug is generated by metabolic or other biological processes after the prodrug is administered. A prodrug may have, relative to the active drug, altered metabolic stability or transport characteristics, fewer side effects or lower toxicity, or improved flavor (for example, see the reference Nogrady, 1985, Medicinal Chemistry A Biochemical Approach, Oxford University Press, New York, pages 388-392, incorporated herein by reference). A prodrug may be synthesized using reactants other than the corresponding drug.

“Solid tumor” refers to solid tumors including, but not limited to, metastatic tumors in bone, brain, liver, lungs, lymph node, pancreas, prostate, skin and soft tissue (sarcoma).

“Therapeutically effective amount” of a drug refers to an amount of a drug that, when administered to a patient with cancer, will have the intended therapeutic effect, e.g., alleviation, amelioration, palliation or elimination of one or more manifestations of cancer in the patient. A therapeutic effect does not necessarily occur by administration of one dose, and may occur only after administration of a series of doses. Thus, a therapeutically effective amount may be administered in one or more administrations.

“Treatment of” a condition or patient refers to taking steps to obtain beneficial or desired results, including clinical results. For purposes of this invention, beneficial or desired clinical results include, but are not limited to, alleviation or improvement of one or more symptoms of cancer; diminishment of extent of disease; delay or slowing of disease progression; alleviation, palliation, or stabilization of the disease state; or other beneficial results. Treatment of cancer may, in some cases, result in partial response or stable disease.

“Tumor cells” refers to tumor cells of any appropriate species, e.g., mammalian such as murine, canine, feline, equine or human.

The term “isotopic variant” refers to a compound that contains an unnatural proportion of an isotope at one or more of the atoms that constitute such compounds. In certain embodiments, an “isotopic variant” of a compound contains unnatural proportions of one or more isotopes, including, but not limited to, hydrogen (¹H) deuterium (²H), tritium (³H), carbon-11 (¹¹C) carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an “isotopic variant” of a compound is in a stable form, that is, non-radioactive. In certain embodiments, an “isotopic variant” of a compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an “isotopic variant” of a compound is in an unstable form, that is, radioactive. In certain embodiments, an “isotopic variant” of a compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I). It will be understood that, in a compound as provided herein, any hydrogen can be ²H, for example, or any carbon can be ¹³C, as example, or any nitrogen can be ¹⁵N, as example, and any oxygen can be ¹⁸O, where feasible according to the judgment of one of skill. In certain embodiments, an “isotopic variant” of a compound contains unnatural proportions of deuterium.

The term “solvate” refers to a complex or aggregate formed by one or more molecules of a solute, e.g., a compound provided herein, and one or more molecules of a solvent, which is present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable.

In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The term “pharmaceutically acceptable excipient” refers to a pharmaceutically-acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response.

### EXAMPLES

### Materials and Methods

### Cell Lines

All human cancer cell lines were obtained from either the American Type Culture Collection (ATCC, Manassas, VA), or Japanese Collection of Research Biosources (JCRB, Osaka Japan) or Cobioer Biosciences (Nanjing, China).

### Reagents and Chemicals

Anti-human AKR1C3 monoclonal antibody, bleomycin, NADPH, lyophilized bovine serum albumin (BSA), and positive control substrates for AKR1C1/AKR1C3 (progesterone, androstenedione, and dihydrotestosterone) were purchased from Sigma (St. Louis, MO). Recombinant human AKR1C3 was purchased from Abcam (Cambridge, MA) and AKR1C1 and AKR1C4 were purchased from Sigma. Comet assay kit was purchased from Trevigen (Gaithersburg, MD). CellTiter Glo (CTG) assay kit was from Promega (Madison, WI). Racemic 2870 was synthesized by Threshold Pharmaceuticals (South San Francisco, CA). Prodrugs 3423 and 3424 were synthesized by Ascentawits Pharmaceuticals, LTD (Shenzhen, China). 3021 was synthesized based on the reported method⁽²⁷⁾. Standard of care therapies were purchased as following: abiraterone (Bos Science, USA), prednisolone (Saen Chemical Technology, China), 5-FU (Shanhai Xudong Haipu Pharmaceutical Co., China), gemcitabine (Vianex S. A., Greece), and sunitinib (Cayman, USA).

In the examples below, the AKR1C3-dependent activation, in vitro 3424 cytotoxicity in a wide range of human cancer cell lines, and concentration-dependent DNA cross-linking of 3424 were investigated. Moreover, we also studied the in vivo anti-tumor activity of 3424 in a broad panel of CDX and PDX models.

### Example 1: AKR1C3-Dependent Activation of 3424

### Enzymatic Activity Assays

The assay mixture consisted of 10-50 μM 3424 or positive control (androstenedione or dihydrotestosterone), 100 mM phosphate buffer, pH 7.0, 300 μM NADPH, 4% ethanol and 8 M recombinant human AKR1C3, AKR1C1 or AKR1C4 to give a total volume of 200 μL. The reaction was incubated at 25° C. and terminated at various time points by adding acetonitrile and methanol (at a ratio of 9 to 1) and subjected for LC-MS/MS analysis. For enzyme kinetic analysis, the activity was determined with a SpectraMax M2^{e} spectrophotometer (Molecular Devices, LLC, San Jose, CA) by measuring the decrease in absorbance of the NADPH at 340 nm (E=6270 M⁻¹ cm⁻¹). After the initiation of the reaction by addition of substrates, the reactions were monitored at 30 s intervals at 25° C. The non-substrate reaction rate was also monitored as background and its slope was used to determine the initial velocity of the reaction. The kinetic data reported were the average of triplicate measurements.

### LC-MS/MS Analyses

For AKR1C3-mediated 3424 metabolism, LC/MS/MS was performed using a Sciex API-4000 Qtrap (ABSciex, LLC, Framingham, MA) mass spectrometer coupled to an Agilent 1200 HPLC system (Agilent Technologies, Santa Clara, CA). For 3424 analysis, reverse phase liquid chromatographic separation was performed with a Waters Xbridge C18 column (2.1×100 mm, 3.5 μm, Waters Corp., Milford, MA) in a total run time of 12 min using a flow rate of 0.3 mL/min. The mobile phase A consisted of 0.1% formic acid in water and mobile phase B consisted of 0.1% formic acid in ACN (acetonitrile). The gradient was performed with an isocratic run at 15% B for 1.5 min and gradient to 50% B at 3 min, then to 95% B at 6 min and holding for 1 min, finally back to 15% B in 0.1 min and equilibrated at 15% B for 4.9 min. The column oven temperature was 40° C. and the sample injection volume was 2 μL. The mass spectra were obtained in positive MRM mode. In positive ion mode, the ion spray voltage was set at 4500 V, declustering potential at 80 V, collision energy at 20 V, source temperature at 350° C., curtain gas at 10 psi and the source gas 1 and 2 both at 60 psi. The MRM pairs for 3424 and 3424-IS were m/z 461->313 and m/z 465->313, respectively. For 2660 analysis, normal phase liquid chromatographic separation was performed with Waters Atlantis HILIC Silica column (2.1 mm×100 mm, 3 μm, Waters Corp., Milford, MA) in a total run time of 9 min using a flow rate of 0.3 mL/min. The mobile phase A consisted of 1 mM ammonium formate in water and mobile phase B using ACN. The gradient was performed from isocratic run at 89% B for 1 min and gradient to 60% B at 1.5 min, then to 40% B at 2.5 min and holding for 2 min, finally back to 89% B in 0.1 min and equilibrated at 15% B for 4.4 min. The column oven temperature was 40° C. and the sample injection volume was 2 μL. The mass spectra were obtained in negative MRM mode. In negative ion mode, the ion spray voltage was set at −4500 V, declustering potential at −60 V, collision energy at −30 V, source temperature at 350° C., curtain gas at 10 psi and the source gas 1 and 2 both at 60 psi. The MRM pairs for 2660 and 2660-IS were m/z 147->63 and m/z 151->63, respectively. The peak area ratio for each MRM transition (peak area of analyte/peak area of analyte-IS) of calibration standards and samples were used for quantitative analysis using Analyst 1.6 software (ABSciex, Framingham, MA).

The activation of 3424 by AKR1C3 was monitored by the reduction of 3424 and the generation of the active form 2660, using LC/MS-MS. As shown in FIG. 1, recombinant human AKR1C3 was able to activate 3424 into 2660 in 60 min (FIGS. 1A and 1B). In contrast, 3424 was not metabolized by AKR1C1 or AKR1C4, two members of the AKR1C family (Table 1). Thus, AKR1C3-dependent activation of prodrug 3424 was evident. AKR1C3 exhibited similar catalytic efficiency towards 3424 (S-enantiomer) and its R-enantiomer 3423 (Table 2). Compared to the physiological substrates 4-androstenedione and 5-ϵ dihydrotestosterone (5′-DHT), 3424 was activated by AKR1C3 at a higher rate.

TABLE 1
Concentration of 2660 (active form) following incubation
of 3424 (prodrug) with various AKR1C enzymes.

| | AKR1C1* | AKR1C4* | AKR1C3 |
|---|---|---|---|
| 3424 | 2660 | 2660 | 2660 |
| Concentration | Concentration | Concentration | Concentration |
| (μM) | (μM) | (μM) | (μM) |
| 0 | BQL | BQL | BQL |
| 10 | — | — | 10.71 ± 0.53 |
| 20 | — | — | 14.77 ± 1.44 |
| 30 | — | — | 33.96 ± 1.90 |
| 40 | — | — | 34.30 ± 6.63 |
| 50 | BQL | BQL | 34.59 ± 7.44 |

n = 3, data expressed as mean ± SD
BQL: below quantification limit (0.25 ng/mL)
*only tested at highest 3424 concentration (50 μM)

TABLE 2
Kinetic analysis of AKR1C3 towards various substrates

| | | Vmax | | Kcat | T_{1/2} | Kcat/Km |
|---|---|---|---|---|---|---|
| Enzyme | Substrate | (μm/min) | Km (μm) | (min⁻¹) | (min) | (min⁻¹mM⁻¹) |
| AKR1C3 | 4-Androstenedione | 0.49 ± 0.04 | 1.98 ± 0.43 | 0.07 ± 0.01 | 165.97 ± 14.69 | 35.35 |
| | 5^{α}-DHT | 0.85 ± 0.01 | 10.18 ± 0.62 | 0.12 ± 0.00 | 95.58 ± 0.99 | 11.79 |
| | 3424 (S form) | 4.45 ± 0.07 | 12.55 ± 0.28 | 0.64 ± 0.01 | 18.17 ± 0.29 | 50.99 |
| | 3423 (R form) | 5.03 ± 0.29 | 12.88 ± 1.71 | 0.72 ± 0.04 | 16.13 ± 0.99 | 59.90 |

Thus, it was confirmed that 3424 reduction is AKR1C3 dependent. Recombinant human AKR1C3, but not AKR1C1 or AKR1C4, reduced 3424 to its active aziridine nitrogen mustard moiety 2660.

### Example 2: AKR1C3-Dependent 3424 Cytotoxicity

### In Vitro Proliferation Assay

Exponentially growing cells were seeded 24 hours before the addition of test compounds. After addition of test compounds, the plates were incubated for the indicated hours at 37° C. in a standard tissue culture incubator. At the end of the experiment, the viable cells were detected using either a CellTiter Glo (CTG) assay kit or AlamarBlue^{(28,29)}. Drug concentration resulting in growth inhibition of 50% (IC₅₀) relative to untreated control was calculated using either XLfit (IDBS, Boston, MA) or Prism 6 (GraphPad, San Diego, CA). For 3021 experiments, cells were pretreated with 3 μM of 3021 for 2 h prior to compound treatment under air. IC₅₀ was calculated as described above.

### Western Blot

Human cell extracts were prepared and protein concentrations were determined. Proteins were detected using antibodies recognizing human AKR1C3 and tubulin, or β-actin. The band densities of AKR1C3 and tubulin or actin were scanned and quantified using the Odyssey laser imaging system and software (LI-COR Biosciences, Lincoln, NE), or UVP ChemStudio imaging system and VisionWorks software (Analytik Jena AG), and the ratio of AKR1C3 to tubulin or actin was calculated.

### Comet Assay

After seeding cells for 24 hours, the test articles were added at the indicated concentrations and incubated for 2 hours. Cells were washed twice to remove compound completely. 20 μmol/L of bleomycin was added and incubated for 1 hour under air to induce DNA strand breaks following 2 h cell resting. After washing twice with PBS, comet assay was conducted using a single-cell electrophoresis system from Trevigen (Gaithersburg, MD). The data were analyzed using Comet Assay IV software from Perceptive Instruments (29).

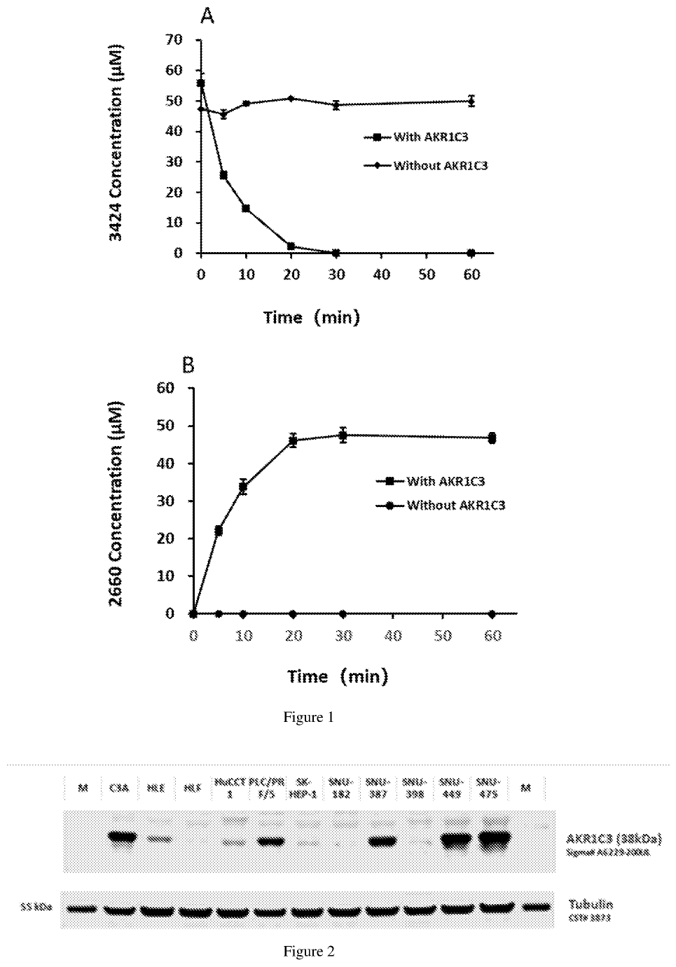

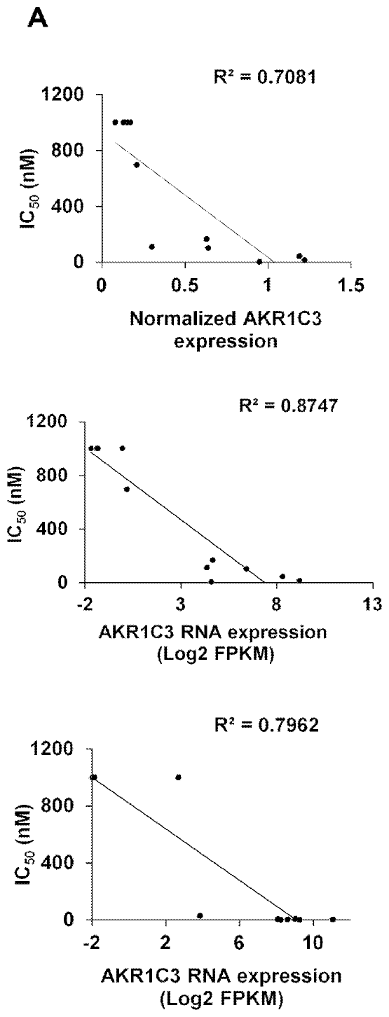

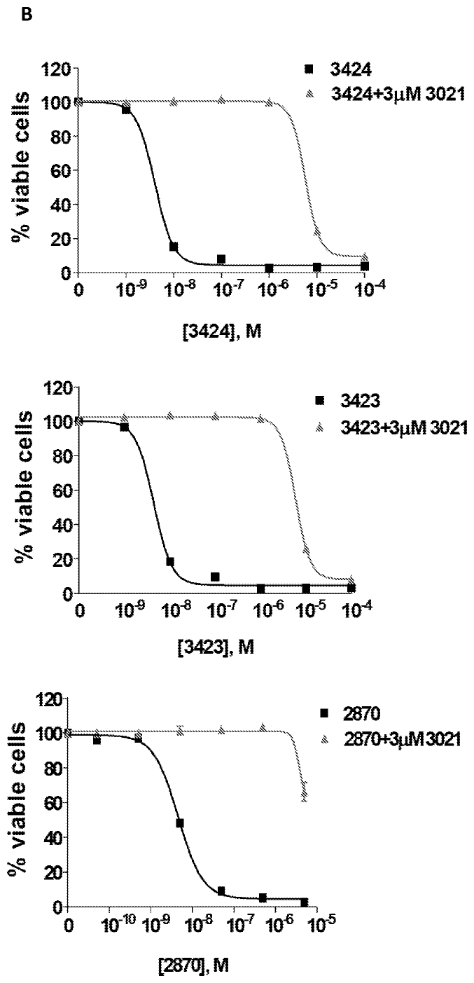

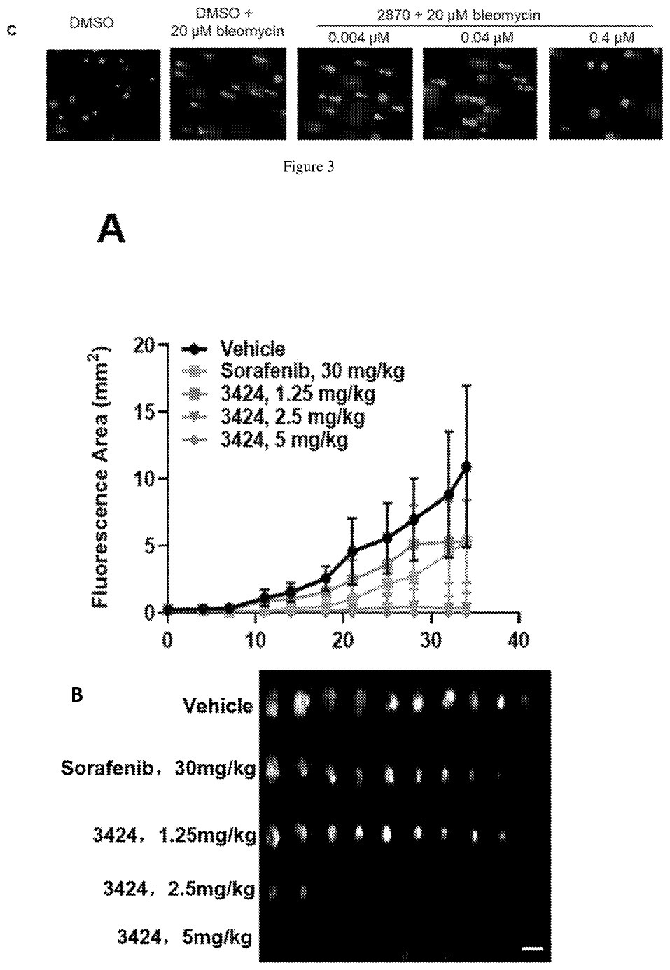

Cytotoxicity of 3424 was evaluated in a panel of liver cancer cell lines and non-small cell lung cancer (NSCLC) cell lines. AKR1C3 protein expression in liver cancer cell lines was determined using Western blot and tubulin was used as a loading control (FIG. 2). AKR1C3 RNA expression data was obtained from the CrownBio (Beijing, China) database. As shown in Table 3, after 96 h exposure to 3424, liver cancer cell lines with high AKR1C3 expression at both the protein and RNA levels were more sensitive to 3424 with IC₅₀ values in a low nanomolar range. On the other hand, cells expressing low AKR1C3 were less sensitive to 3424 with IC₅₀ values higher than 1000 nM. Similarly, NSCLC cells also exhibited an AKR1C3-dependent cytotoxic profile after 72 h exposure to 3424 (Table 4). There was a high correlation between 3424 IC₅₀ and the level of AKR1C3 protein (R²=0.71, FIG. 3A, left) and RNA expression (R²=0.87, FIG. 3A, middle) in liver cancer cells and in NSCLC cells (R²=0.80, FIG. 3A, right), respectively. These results demonstrated that 3424-mediated cytotoxicity was highly correlated with the level of AKR1C3 expression in both liver cancer and NSCLC cell lines.

TABLE 3
3424 cytotoxicity against a panel of human liver
cancer cell lines after 96 hours of drug exposure

| | | AKR1C3 | AKR1C3 RNA | |
|---|---|---|---|---|
| | Normalized | protein | expression | 3424 |
| Liver cancer | AKR1C3 | expression | level | IC₅₀ |
| cell line | expression | level | (Log2 FPKM) | (nM) |
| SNU-475 | 1.22 | high | 9.19 | 15 |
| SNU-449 | 1.19 | high | 8.3 | 45 |
| C3A | 0.95 | high | 4.58 | 5 |
| SNU-387 | 0.64 | Medium | 6.42 | 103 |
| PLC/PRF/5 | 0.63 | Medium | 4.66 | 167 |
| HLE | 0.3 | Medium | 4.36 | 113 |
| HuCCT1 | 0.21 | Low | 0.19 | 696 |
| SNU-182 | 0.17 | Low | −0.05 | >1000 |
| HLF | 0.15 | Low | −1.69 | >1000 |
| SK-HEP-1 | 0.13 | Low | −1.39 | >1000 |
| SNU-398 | 0.08 | Low | −1.33 | >1000 |

TABLE 4
3424 cytotoxicity against a panel of human non-small cell lung
cancer cell lines (NSCLC) after 72 hours of drug exposure

| | AKR1C3 RNA | 3424 |
|---|---|---|
| NSCLC cancer | expression level | IC₅₀ |
| cell line | (Log2 FPKM) | (nM) |
| NCI-H1944 | 11.06 | 2.3 |
| NCI-H2228 | 9.25 | 0.21 |
| NCIH1755 | 9 | 8.2 |
| NCI-H1563 | 8.61 | 2.5 |
| NCI-H2110 | 8.23 | 1.1 |
| NCI-H1792 | 8.07 | 4.5 |
| CAL12T | 3.86 | 29.1 |
| NCIH2106 | 2.68 | >1000 |
| NCI-H23 | −1.98 | >1000 |
| NCI-H522 | −1.88 | >1000 |

AKR1C3-mediated specific activation of 3424 was confirmed using the AKR1C3 inhibitor 3021 in H460 cells. After 2 h pretreatment of H460 cells with 3 μM 3021 followed by co-treatment with 3424 for 2 hours, the AKR1C3-specific inhibitor, 3021, was able to effectively inhibit 3424 cytotoxicity in H460 with an IC₅₀ of 6.3 μM as compared to an IC₅₀ of 4 nM in the absence of 3021 (FIG. 3B, left), which was also reported by Evans et al. (²⁵). Here we also profiled the cytotoxicity of the R-enantiomer, 3423, and a racemic mixture of R- and S-enantiomers at 1:1 ratio, 2870, in the absence or presence of 3021 in H460 cells. As shown in FIG. 3B, 3423 (middle) and 2870 (right) exerted equally potent cytotoxicity to 3424 with IC₅₀ values of 5 nM and 4 nM, respectively. Similar to 3424, the cytotoxicity of 3423 and 2870 was highly AKR1C3-dependent and 3021 inhibited their cytotoxicity at concentrations over 1000-fold greater than in the absence of the inhibitor with IC₅₀ values of 6.3 μM and >5 μM, respectively. Inhibitor 3021 alone did not exert any cytotoxicity up to 100 μM (data not shown).

To evaluate whether 3424 cross-linked DNA, a direct biochemical assay for DNA cross-linking, the single-cell electrophoresis-based comet assay was employed using H460 cells. In this assay, the racemic mixture, 2870, was used. As shown in FIG. 3C, there was no detectable double-strand breaks with tail moment 0.3 in vehicle DMSO-treated H460 cells. Bleomycin was used to induce DNA strand breaks which was evident by increased tail moment to 35. 2870-induced DNA cross-linking was tested using three concentrations under identical conditions that were used for bleomycin. Compound 2870-induced concentration-dependent DNA-crosslinking was evident by a decreased tail moment from 36 to 1.6.

Thus, it was demonstrated that 3424-mediated cytotoxicity is highly AKR1C3-dependent.

### In Vivo Anti-Tumor Activity

All procedures related to animal handling, care, and the treatment in this study were performed according to guidelines approved by the Institutional Animal Care and Use Committee (IACUC) of CrownBio (Beijing, China), WuXi AppTec (Shanghai, China), or Eurofins Pharmacology Discovery Services Taiwan (Taipei, Taiwan) following the guidance of the Association for Assessment and Accreditation of Laboratory Animal Care (AAALAC). At the time of routine monitoring, the animals were checked for any effects of tumor growth on normal behavior such as mobility, food and water consumption, body weight gain/loss, eye/hair matting and any other abnormal effect. Death and observed clinical signs were recorded on the basis of the numbers of animals within each subset.

For all the animal studies, drug efficacy was assessed by tumor growth inhibition. Tumor volume (mm³) was measured twice weekly following the prolate ellipsoid formula: Length (mm)×[Width (mm)]²×0.5. Percent Tumor Growth Inhibition (% TGI) was determined twice weekly during the dosing period by the formula: % TGI=(1−[(T−T0)/(C−C0)])×100 where T=mean tumor volume of treated group, T0=mean tumor volume of treated group at study start, C=mean tumor volume of control group and C0=mean tumor volume of control group at study start. Two-way ANOVA and Bonferroni test were used to assess the statistical significance of groups compared to respective vehicle control using SPSS Statistics 23 (IBM, Armonk, NY) or R (version 3.3.1). P-values of <0.05 were regarded as statistically significant.

### Example 3: Anti-Tumor Efficacy of 3424 in CDX Models

### Example 3-1: Anti-Tumor Efficacy of 3424 in HepG2 and H460 Models

In vivo anti-tumor activity of 3424 was evaluated using GFP-expressing cancer cell lines in an orthotopic liver cancer model (HepG2) and a subcutaneous lung cancer model (H460) CDX models at AntiCancer, Inc (Beijing, China). Female athymic nude mice (6 weeks; BALB/c-nu, Beijing HFK Bioscience Co., Ltd., Beijing, China) were used in the studies. Each mouse was implanted with a HepG2-GFP tumor chunk (˜1 mm³ in diameter) in the right lobe of the liver for tumor development or inoculated subcutaneously with H460-GFP tumor chunk (˜1 mm³ in diameter). About 3-10 days later, whole mouse body scan was performed using FluorVivo Model-100 fluorescence imager (INDEC Biosystems, Inc., Los Altos, CA). Mice with similar fluorescent areas were selected and randomly grouped. Both cell lines expressed high level of AKR1C3. Prodrug 3424 was dosed intravenously (IV) at 1.25, 2.5 mg/kg or 5 mg/kg Q7D×2 for the HepG2 orthotopic model or at 0.625, 1.25 or 2.5 mg/kg for the H460 xenograft model with a regimen of Q7D×2, 1 week off, then Q7D×2 again. Sorafenib was used as the positive control in the HepG2 model and was dosed orally at 30 mg/kg with a regimen of QD×5×7 cycles. Taxol was used as the positive control in the H460 model and was administered at 15 mg/kg IV with a regimen of BIW×4. During the experiments, mice were observed daily and body weight was measured twice a week. Tumor burden was monitored twice a week either by caliper measurement (H460) or FluorVivo fluorescence imager (H460-GFP and HepG2-GFP).

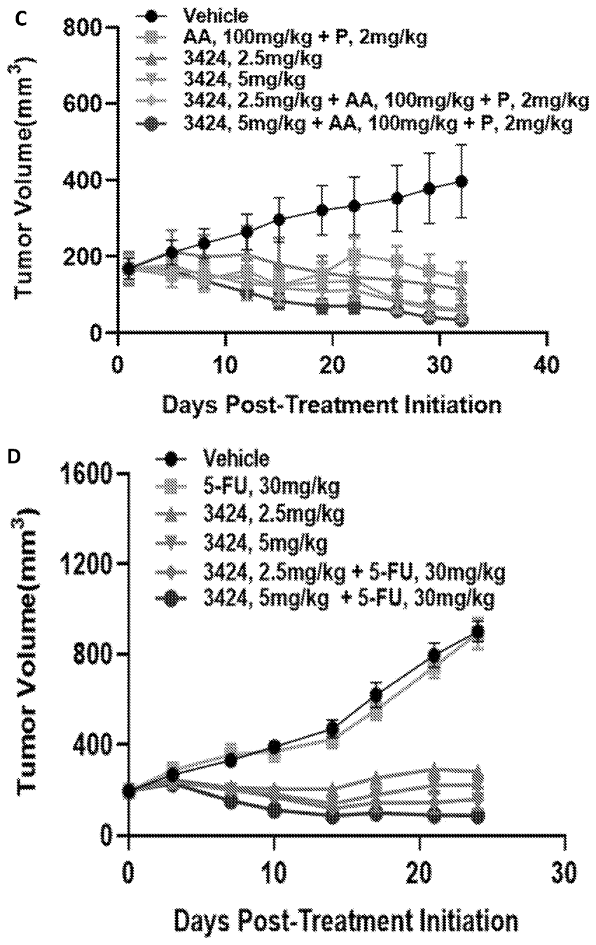

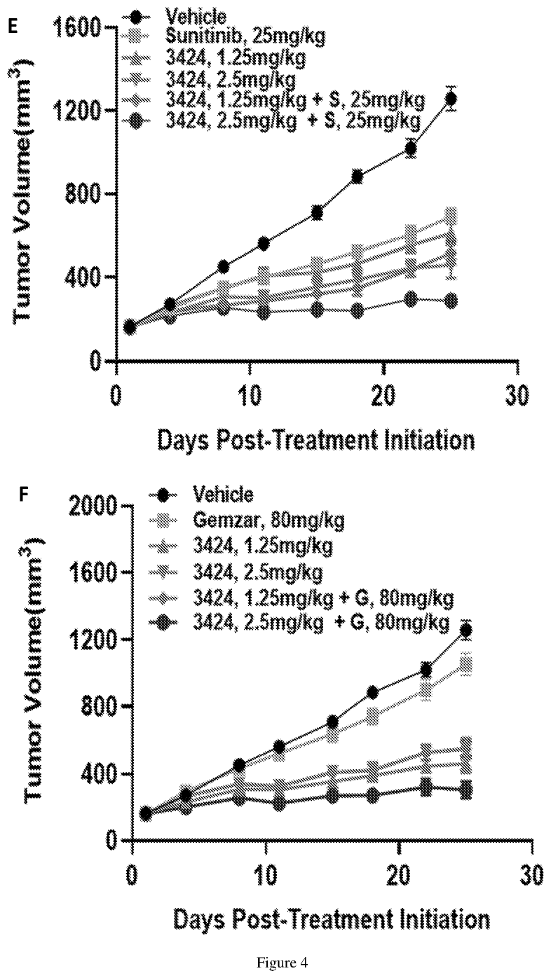

Using the HepG2 orthotopic xenograft model, we investigated the dose-dependent anti-tumor activity of 3424 by whole body fluorescence imaging. When 3424 was administered via IV injection at doses of 1.25, 2.5 or 5 mg/kg once a week for 2 weeks, the tumor growth inhibition (TGI) at Day 34 was 52.4%, 91.5% and 101.2%, respectively (Table 5). The anti-tumor efficacy of 3424 was observed in a dose-dependent manner (FIG. 4A). Compared to the vehicle group, tumor inhibition induced by 3424 at 2.5 mg/kg and 5 mg/kg was statistically significant with a complete regression rate of 80% and 100%, respectively. At the end of the experiment, the data from tumor fluorescent images (FIG. 4B) and tumor weight were consistent with the measurement of fluorescence area (FIG. 4A). Sorafenib, a first-line treatment of hepatocellular carcinoma (HCC), was administered orally at 30 mg/kg and reduced tumor growth by 52.1% with no statistical significance. With this dosing regimen, sorafenib resulted in body weight loss (−11%, data not shown). In contrast, no body weight loss was observed in 3424-treated groups at all tested doses in the current study (Table 5).

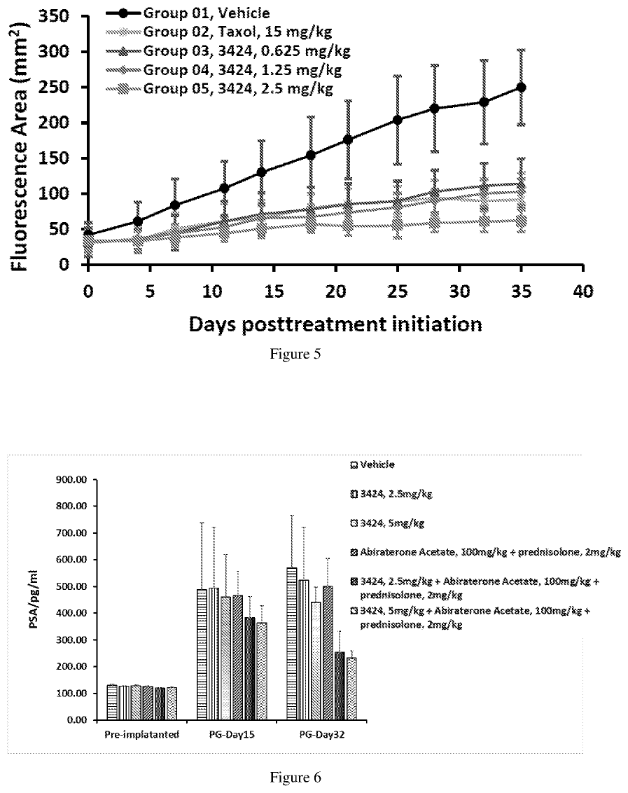

In the H460 subcutaneous model, prodrug 3424 was given weekly for 2 doses; with one week off, and another 2 weekly doses at 0.625, 1.25, and 2.5 mg/kg. As shown in FIG. 5 and Table 5, prodrug 3424 exhibited dose-dependent anti-tumor activity with TGI of 60.2%, 67.2% and 88%, respectively. The anti-tumor efficacy of 3424 was comparable to paclitaxel, with paclitaxel at 15 mg/kg showing a TGI of 64%. At the end of the study (Day 35), the treatment of 3424 resulted in minimal body weight loss at low- and mid-dose and a statistically insignificant 14% loss at the high dose, whereas paclitaxel treatment caused a significant 11% body weight loss (Table 5).

TABLE 5
Summary of 3424 in vivo efficacy in HepG2 and H460 CDX models

| | | | | | | | | AKR1C3 |
|---|---|---|---|---|---|---|---|---|
| CDX | Cancer | | | | 3424 Dose | TGI | Body weight | LOG2 |
| model | type | Implantation | Route | Regimen | (mg/kg) | % | change % | (FPKM) |
| HepG2 | Liver | Orthotopic | i.v. | Q7D × 2 | 1.25 | 52.4 | 22.8 | 6.9 |
| | | | | | 2.5 | 91.5 | 19.5 | |
| | | | | | 5 | 101.2 | 17.1 | |
| | | | | Q7D × 2; | 0.625 | 60.2 | −1 | |
| H460 | Lung | Subcutaneous | i.v. | 1 week off; | 1.25 | 67.2 | −1 | 8.6 |
| | | | | Q7D × 2 | 2.5 | 88.0 | −14 | |

### Example 3-2: Anti-Tumor Efficacy of 3424 in VCaP, SNU-16 and A498 Models

The anti-tumor activities of 3424 and the compositions comprising it were evaluated in castration-resistant prostate cancer (CRPC) VCaP, gastric cancer SNU-16, and renal cell carcinoma (RCC) A498 xenograft models. Studies were conducted at WuXi AppTec (VCaP and SNU-16 models) and Eurofins Pharmacology Discovery Services Taiwan (A498 model). Male BALB/c nude, female BALB/c nude and female SCID mice were used in the CDX models of CRPC, gastric cancer, and RCC, respectively. Human cancer cells at 5×10⁶ or 1×10⁷ with 1:1 matrigel were injected subcutaneously into the right flank of mice. Vehicle and test articles were administered when tumor volumes reached 150-200 mm³ (denoted as Day 1, or Day 0 in SNU-16 model). Vehicle or test articles were administered intravenously once weekly for a total of 4 or 5 doses dependent on the model. Standard of care therapies including abiraterone/prednisolone (for CRPC), 5-fluorouracil (for gastric cancer), and sunitinib and gemcitabine (both for RCC) were administered as recommended in the literature^{(15,30-32)}. On days of co-administration, IV injection of 3424 was done first, followed by the combined agent within 1 hour.

In vivo anti-tumor efficacy of 3424 as a monotherapy, or in combination with standard of care therapy, was evaluated in castration-resistant prostate cancer (CRPC) VCaP, gastric cancer SNU-16, and renal cell carcinoma A498 xenograft models. These three human cancer cell lines expressed high levels of AKR1C3 at both levels of protein and RNA. AKR1C3 protein expression in VCap, SNU-16 and A498 was determined by Western Blot with a ratio of AKR1C3 to tubulin at 8.9, 1.9 and 1.6, respectively. AKR1C3 RNA expression (LOG2 FPKM) in VCap, SNU-16 and A498 was 5.2, 8.0 and 10.0, respectively.

In those studies, animals were treated with various concentrations of 3424 as a monotherapy, an anti-cancer drug or drugs as a monotherapy (standard of care therapies, control), or the compositions of the present invention which combine 3424 and at least one anti-cancer drug.

### Example 3-2-1: 3424 as a Monotherapy or in Combination with Abiraterone Acetate+Prednisolone

In the CRPC model, castrated male BALB/c nude mice were treated with 3424 (weekly IV injection for 5 doses), abiraterone acetate plus prednisolone (daily oral gavage), or the combination (FIG. 4C). At 5 mg/kg, 3424 showed significant TGI of 148%, which was further enhanced to 158% when combined with abiraterone acetate/prednisolone.

Furthermore, at terminal sacrifice on Day 32, animals receiving the combination showed a corresponding reduction in serum prostate specific antigen (PSA) (FIG. 6).

### Example 3-2-2: 3424 as a Monotherapy or in Combination with 5-FU

In the gastric cancer SNU-16 model, female BALB/c mice were treated with 3424 (weekly IV injection for 4 doses), 5-flurouracie (5-FU) (IP injection, twice a week), or 3424 combined with 5-FU (FIG. 4D). Animals treated with 3424 at 2.5 or 5 mg/kg showed remarkable TGI of 87.8% or 96.2%, respectively, whereas 5-FU had no anti-tumor activity at the level of 30 mg/kg. Synergistic effect was noted when 3424 was combined with 5-FU.

### Example 3-2-3: 3424 as a Monotherapy or in Combination with Sunitinib

In the renal cell carcinoma A498 model, female SCID mice were administered 3424 (weekly IV injection for 4 doses), sunitinib (25 mg/kg, daily oral gavage), or the combination (FIG. 4E). At 2.5 mg/kg, 3424 showed a significant TGI of 73%, compared to a modest 52% TGI by sunitinib. When animals were treated with a combination of 3424 and sunitinib, the TGI was further increased to 88%.

### Example 3-2-4: 3424 as a Monotherapy or in Combination with Gemcitabine (Gemzar)

The efficacy of 3424 combined with gemcitabine was also tested in A498 xenograft model, where gemcitabine at 80 mg/kg (weekly IP injection for 4 doses) offered 19% TGI only but in combination with 2.5 mg/kg 3424, the TGI was increased to 87% (FIG. 4F).

In all three models, 3424 was well tolerated in mice and there was no significant body weight loss during the treatment (data not shown).

FIG. 4 depicts the anti-tumor activity of 3424 or the compositions of the present invention in various CDX models. In combination therapy, we have demonstrated that 3424 could enhance the efficacy of the standard of care in the CDX models of CRPC, gastric cancer, and RCC.

### Example 4: Anti-Tumor Activity of 3424 in PDX Models

Anti-tumor activity of 3424 in PDX models was assessed at CrownBio Bioscience (Beijing, China) Inc. using female BALB/c nude mice (6-7 weeks old, Beijing Anikeeper Biotech Co., Ltd, Beijing, China). Tumor fragments (PA1280, GA6201, LU2057 and LU2505) from stock mice inoculated with selected primary human cancer tissues (pancreatic, gastric cancer, and lung) were harvested and used for inoculation into BALB/c nude mice. Each mouse was inoculated subcutaneously for tumor development. Mice were allocated randomly into experimental groups when the average tumor size reached ˜100 mm³) by using StudyDirector™ Ver 3.1.399.19 (Studylog Systems, Inc., S. San Francisco, CA, USA). Prodrug 3424 was administered IV at the indicated doses with a regimen of Q7D×3. Each group consisted of 5-6 mice. The grouping day was denoted as Day 0. Prodrug 3424 was administrated to the tumor-bearing mice from Day 0 through Days as indicated for each study.

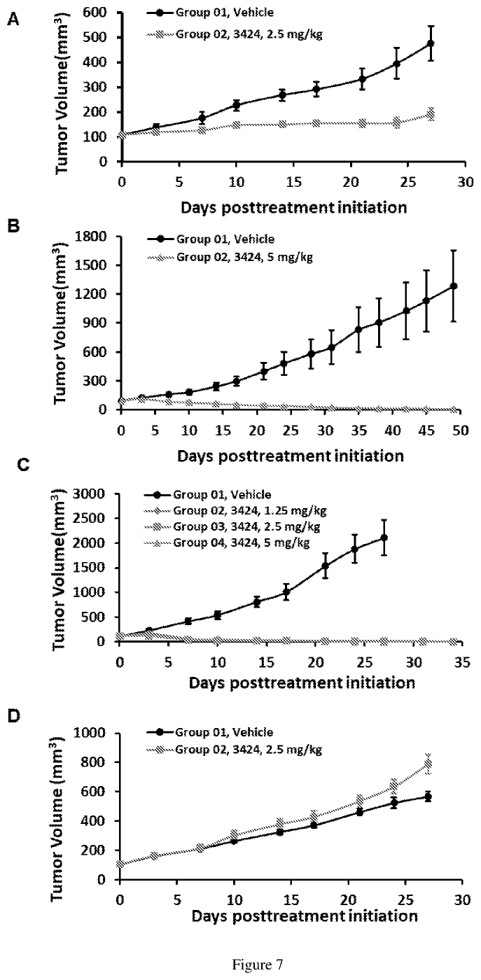

In vivo anti-tumor activities of 3424 were further evaluated in a panel of patient-derived xenograft (PDX) models, including pancreatic cancer, gastric cancer, and two lung cancers, one with high AKR1C3 expression and the other with low AKR1C3 expression. Prodrug 3424 was administered weekly for 3 doses via IV injection. Patient information for the four PDX models is shown in Table 6. In the pancreatic PDX model (FIG. 7A and Table 7), 3424 at 2.5 mg/kg exhibited statistically significant anti-tumor activity of 77.4% TGI. In the gastric PDX model, 5 mg/kg 3424 displayed a remarkable anti-tumor inhibition of 110% TGI (FIG. 7B and Table 7). Tumor volume continued to decrease and remained low or unmeasurable even 1 month after discontinuation of therapy. At the end of the study, 60% of mice remained tumor free. Two lung cancer PDX models with differential levels of AKR1C3 expression were chosen to study if the in vivo anti-tumor activity of 3424 is AKR1C3-dependent. LU2505 PDX model expressed higher AKR1C3 RNA (log 2=6.03) whereas LU2057 model expressed low AKR1C3 RNA (log 2=2.08). As shown in FIGS. 7C and 7D and Table 7, at the same dose of 2.5 mg/kg, 3424 exerted excellent tumor growth inhibition in LU2505 with high AKR1C3 expression (TGI 105.2%) but no inhibition was observed in LU2057 PDX tumor (TGI −20.9%) with low AKR1C3 expression, indicating an AKR1C3-dependent in vivo anti-tumor activity of 3424. Even at a lower dose of 1.25 mg/kg, 3424 still exhibited statistically significant inhibition of LU2505 tumor growth (TGI 105.0%). In all PDX models, prodrug 3424 was well-tolerated with no body weight loss observed at all tested doses (Table 7).

TABLE 6
PDX patient information

| | Patient information | | | | | |
|---|---|---|---|---|---|---|
| HuPrime | | | | | | Pathology |
| Model ID | Cancer type | Subtype | Race | Gender | Age | diagnosis |
| LU2057 | Lung cancer | SCLC | Western | M | NA | Lung cancer |
| LU2505 | Lung cancer | NSCLC, ADC | Asian | F | 69 | Lung cancer |
| PA1280 | Pancreatic cancer | ADC | Asian | F | 82 | Pancreatic cancer |
| GA6201 | Gastric cancer | ADC | Asian | M | 60 | Gastric cancer |

TABLE 7
Summary of 3424 in vivo efficacy in PDX models

| | | | | | | | Body | |
|---|---|---|---|---|---|---|---|---|
| | | | | | 3424 | | weight | AKR1C3 |
| PDX | Cancer | | | | Dose | TGI | change | LOG2 |
| model | type | Implantation | Route | Regimen | (mg/kg) | % | (%) | (FPKM) |
| PA1280 | Pancreas | Subcutaneous | i.v. | Q7D × 3 | 2.5 | 77.4 | 4.72 | 9.04 |
| GA6201 | Gastric | Subcutaneous | i.v. | Q7D × 3 | 5 | 110 | 4 | 6.78 |
| LU2057 | Lung | Subcutaneous | i.v. | Q7D × 3 | 2.5 | −20.9 | 7.69 | 2.08 |
| | | | | | 1.25 | 105.0 | −0.54 | |
| LU2505 | Lung | Subcutaneous | i.v. | Q7D × 3 | 2.5 | 105.2 | 3.37 | 6.03 |
| | | | | | 5 | 105.2 | 1.1 | |

By using more than 20 cell lines from either liver cancer or NSCLC, we found that 3424 in all cell lines with high expression of AKR1C3 exhibited enhanced cytotoxicity compared to cells expressing low or no detectable AKR1C3. The IC₅₀ values of 3424 in cell lines expressing high AKR1C3 were in the low nanomolar range, indicating a high potency that is characteristic of a potent nitrogen mustard. Of particular note was the finding of an enhanced cytotoxicity up to 5000-fold in NCI-H2228 NSCLC line expressing high AKR1C3. This result is consistent with targeting tumors with high expression of AKR1C3 while sparing low AKR1C3 expressing regions found in normal tissues.

The results described herein highlight that 3424 exhibits AKR1C3-dependent cytotoxicity in vitro and anti-tumor activity in vivo in a wide range of human cancer types. We show excellent in vivo anti-tumor activity of 3424 at clinically achievable doses in a broad panel of CDX and PDX models with high expression of AKR1C3. Of note, 3424 shows remarkable in vivo efficacy towards liver, gastric, kidney, lung, pancreatic, and castration-resistant prostate cancers. The AKR1C3-dependent activity of 3424 has served as the basis for ongoing and future clinical trials that target cancer cells specifically and as a biomarker to profile cancer patients and further guide patient selection for therapy with 3424.

The above description of embodiments of the present invention does not limit the present invention. Those skilled in the art can make various modifications and changes according to the present invention, and any modification and change within the spirit of the present invention shall be covered in the scope of the claims appended to the present invention.

All references cited herein are incorporated herein by reference to the full extent allowed by law. The discussion of those references is intended merely to summarize the assertions made by their authors. No admission is made that any reference (or a portion of any reference) is relevant prior art. Applicants reserve the right to challenge the accuracy and pertinence of any cited reference.

### REFERENCES

1. Lin H K, Jez J M, Schlegel B P, Peehl D M, Pachter J A, Penning T M. Expression and characterization of recombinant type 2 3 alpha-hydroxysteroid dehydrogenase (HSD) from human prostate: demonstration of bifunctional 3 alpha/17 beta-HSD activity and cellular distribution. Mol Endocrinol 1997; 11:1971-84
2. Dufort I, Rheault P, Huang X F, Soucy P, Luu—The V. Characteristics of a highly labile human type 5 17beta-hydroxysteroid dehydrogenase. Endocrinology 1999; 140:568-74
3. Penning T M, Drury J E. Human aldo-keto reductases: Function, gene regulation, and single nucleotide polymorphisms. Arch Biochem Biophys 2007; 464:241-50
4. Rizner T L, Penning T M. Role of aldo-keto reductase family 1 (AKR1) enzymes in human steroid metabolism. Steroids 2014; 79:49-63
5. Chang T S, Lin H K, Rogers K A, Brame L S, Yeh M M, Yang Q, et al. Expression of aldo-keto reductase family 1 member C3 (AKR1C3) in neuroendocrine tumors & adenocarcinomas of pancreas, gastrointestinal tract, and lung. Int J Clin Exp Pathol 2013; 6:2419-29
6. Guise C P, Abbattista M R, Singleton R S, Holford S D, Connolly J, Dachs G U, et al. The bioreductive prodrug PR-104A is activated under aerobic conditions by human aldo-keto reductase 1C3. Cancer Res 2010; 70:1573-84
7. Miller V L, Lin H K, Murugan P, Fan M, Penning T M, Brame L S, et al. Aldo-keto reductase family 1 member C3 (AKR1C3) is expressed in adenocarcinoma and squamous cell carcinoma but not small cell carcinoma. Int J Clin Exp Pathol 2012; 5:278-89
8. Zhao J, Zhang M, Liu J, Liu Z, Shen P, Nie L, et al. AKR1C3 expression in primary lesion rebiopsy at the time of metastatic castration-resistant prostate cancer is strongly associated with poor efficacy of abiraterone as a first-line therapy. Prostate 2019; 79:1553-62
9. Nakarai C, Osawa K, Akiyama M, Matsubara N, Ikeuchi H, Yamano T, et al. Expression of AKR1C3 and CNN3 as markers for detection of lymph node metastases in colorectal cancer. Clin Exp Med 2015; 15:333-41
10. Hsu N Y, Ho H C, Chow K C, Lin T Y, Shih C S, Wang L S, et al. Overexpression of dihydrodiol dehydrogenase as a prognostic marker of non-small cell lung cancer. Cancer Res 2001; 61:2727-31
11. Powell K, Semaan L, Conley-LaComb M K, Asangani I, Wu Y M, Ginsburg K B, et al. ERG/AKR1C3/A R Constitutes a Feed-Forward Loop for A R Signaling in Prostate Cancer Cells. Clin Cancer Res 2015; 21:2569-79
12. Heibein A D, Guo B, Sprowl J A, Maclean D A, Parissenti A M. Role of aldo-keto reductases and other doxorubicin pharmacokinetic genes in doxorubicin resistance, DNA binding, and subcellular localization. BMC Cancer 2012; 12:381
13. Zhong T, Xu F, Xu J, Liu L, Chen Y. Aldo-keto reductase 1C3 (AKR1C3) is associated with the doxorubicin resistance in human breast cancer via PTEN loss. Biomed Pharmacother 2015; 69:317-25
14. Liu C, Lou W, Zhu Y, Yang J C, Nadiminty N, Gaikwad N W, et al. Intracrine Androgens and AKR1C3 Activation Confer Resistance to Enzalutamide in Prostate Cancer. Cancer Res 2015; 75:1413-22
15. Liu C, Armstrong C M, Lou W, Lombard A, Evans C P, Gao A C. Inhibition of AKR1C3 Activation Overcomes Resistance to Abiraterone in Advanced Prostate Cancer. Mol Cancer Ther 2017; 16:35-44
16. Zhao J, Xiang Y, Xiao C, Guo P, Wang D, Liu Y, et al. AKR1C3 overexpression mediates methotrexate resistance in choriocarcinoma cells. Int J Med Sci 2014; 11:1089-97
17. Xiong W, Zhao J, Yu H, Li X, Sun S, Li Y, et al. Elevated expression of AKR1C3 increases resistance of cancer cells to ionizing radiation via modulation of oxidative stress. PLoS One 2014; 9:e111911
18. Sun S Q, Gu X, Gao X S, Li Y, Yu H, Xiong W, et al. Overexpression of AKR1C3 significantly enhances human prostate cancer cells resistance to radiation. Oncotarget 2016; 7:48050-8
19. Xie L, Yu J, Guo W, Wei L, Liu Y, Wang X, et al. Aldo-keto reductase 1C3 may be a new radioresistance marker in non-small-cell lung cancer. Cancer Gene Ther 2013; 20:260-6
20. Ascierto M L, McMiller T L, Berger A E, Danilova L, Anders R A, Netto G J, et al. The Intratumoral Balance between Metabolic and Immunologic Gene Expression Is Associated with Anti-PD-1 Response in Patients with Renal Cell Carcinoma. Cancer Immunol Res 2016; 4:726-33
21. Moradi Manesh D, El-Hoss J, Evans K, Richmond J, Toscan C E, Bracken L S, et al. AKR1C3 is a biomarker of sensitivity to PR-104 in preclinical models of T-cell acute lymphoblastic leukemia. Blood 2015; 126:1193-202
22. Guise C P, Abbattista M R, Tipparaju S R, Lambie N K, Su J, Li D, et al. Diflavin oxidoreductases activate the bioreductive prodrug PR-104A under hypoxia. Mol Pharmacol 2012; 81:31-40
23. Guise C P, Wang A T, Theil A, Bridewell D J, Wilson W R, Patterson A V. Identification of human reductases that activate the dinitrobenzamide mustard prodrug PR-104A: a role for NADPH:cytochrome P450 oxidoreductase under hypoxia. Biochem Pharmacol 2007; 74:810-20
24. Patterson A V, Ferry D M, Edmunds S J, Gu Y, Singleton R S, Patel K, et al. Mechanism of action and preclinical antitumor activity of the novel hypoxia-activated DNA cross-linking agent PR-104. Clin Cancer Res 2007; 13:3922-32
25. Evans K, Duan J, Pritchard T, Jones C D, McDermott L, Gu Z, et al. OBI-3424, a Novel AKR1C3-Activated Prodrug, Exhibits Potent Efficacy against Preclinical Models of T-ALL. Clin Cancer Res 2019; 25:4493-503
26. Wang Y, Liu Y, Zhou C, Wang C, Zhang N, Cao D, et al. An AKR1C3-specific prodrug with potent anti-tumor activities against T-ALL. Leuk Lymphoma 2020:1-9
27. Flanagan J U, Atwell G J, Heinrich D M, Brooke D G, Silva S, Rigoreau L J, et al. Morpholylureas are a new class of potent and selective inhibitors of the type 5 17-beta-hydroxysteroid dehydrogenase (AKR1C3). Bioorg Med Chem 2014; 22:967-77
28. Meng F, Bhupathi D, Sun J D, Liu Q, Ahluwalia D, Wang Y, et al. Enhancement of hypoxia-activated prodrug TH-302 anti-tumor activity by Chkl inhibition. BMC Cancer 2015; 15:422
29. Meng F, Evans J W, Bhupathi D, Banica M, Lan L, Lorente G, et al. Molecular and cellular pharmacology of the hypoxia-activated prodrug TH-302. Mol Cancer Ther 2012; 11:740-51
30. Lee Y, Vassilakos A, Feng N, Jin H, Wang M, Xiong K, et al. GTI-2501, an antisense agent targeting R1, the large subunit of human ribonucleotide reductase, shows potent anti-tumor activity against a variety of tumors. Int J Oncol 2006; 28:469-78
31. Oksala R, Moilanen A, Riikonen R, Rummakko P, Karjalainen A, Passiniemi M, et al. Discovery and development of ODM-204: A Novel nonsteroidal compound for the treatment of castration-resistant prostate cancer by blocking the androgen receptor and inhibiting CYP17A1. J Steroid Biochem Mol Biol 2019; 192:105115
32. Wang X, Zhang L, O'Neill A, Bahamon B, Alsop D C, Mier J W, et al. Cox-2 inhibition enhances the activity of sunitinib in human renal cell carcinoma xenografts. Br J Cancer 2013; 108:319-26
33. Spiegelberg L, Houben R, Niemans R, de Ruysscher D, Yaromina A, Theys J, et al. Hypoxia-activated prodrugs and (lack of) clinical progress: The need for hypoxia-based biomarker patient selection in phase III clinical trials. Clin Transl Radiat Oncol 2019; 15:62-9
34. Heeke A L, Pishvaian M J, Lynce F, Xiu J, Brody J R, Chen W J, et al. Prevalence of Homologous Recombination-Related Gene Mutations Across Multiple Cancer Types. JCO Precis Oncol 2018; 2018

1. (canceled)

2. (canceled)

3. (canceled)

4. (canceled)

5. A method for treating cancer in a patient in need thereof, comprising the step of administering to the patient an effective amount of the compound 1-(3-(3-N,N-dimethylaminocarbonyl)phenoxyl-4-nitrophenyl)-1-ethyl-N,N′-bis(ethylene)phosphoramidate having the following Formula I, or a pharmaceutically acceptable salt, isotopic variant or solvate thereof 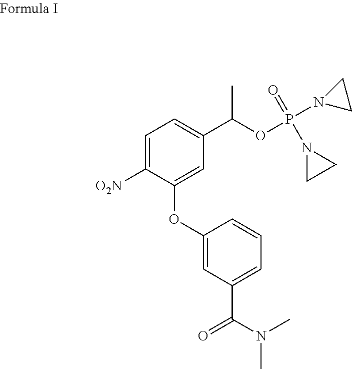 [SMILES: CC(OP(=O)(N1CC1)N1CC1)c1ccc([N+](=O)[O-])c(Oc2cccc(C(=O)N(C)C)c2)c1]
wherein the AKR1C3 reductase level of the cancer is represented by the AKR1C3 protein level or RNA level and is equal to or greater than a predetermined value.

6. The method according to claim 5, wherein the compound is (S)-1-(3-(3-N,N-dimethylaminocarbonyl)phenoxyl-4-nitrophenyl)-1-ethyl-N,N′-bis(ethylene)phosphoramidate having the following Formula I-1, or (R)-1-(3-(3-N,N-dimethylaminocarbonyl)phenoxyl-4-nitrophenyl)-1-ethyl-N,N′-bis(ethylene)phosphoramidate having the following Formula 1-2

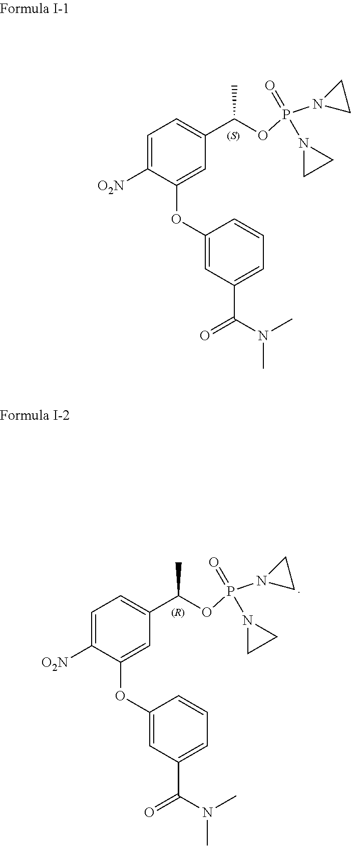

7. The method according to claim 5, wherein the cancer is liver cancer, hepatocellular carcinoma (HCC), lung cancer, melanoma, prostate cancer, breast cancer, leukemia, esophageal cancer, renal cancer, gastric cancer, colon cancer, brain cancer, bladder cancer, cervical cancer, ovarian cancer, head and neck cancer, endometrial cancer, pancreatic cancer, a sarcoma cancer, or rectal cancer.

8. The method according to claim 7, wherein the cancer is liver cancer, non-small cell lung cancer, castrate-resistant prostate cancer, gastric cancer, renal cell carcinoma or pancreatic cancer.

9. The method according to claim 5, wherein the method further comprises a step for measuring the content of AKR1C3 reductase of cancer cells in a patient using AKR1C3 antibodies, where the content of AKR1C3 reductase is measured to be equal to or greater than the predetermined value, the compound is administered to the patient.

10. A method for inhibiting the growth of a cell, comprising the step of contacting the cell with an effective amount of compound 1-(3-(3-N,N-dimethylaminocarbonyl)phenoxyl-4-nitrophenyl)-1-ethyl-N,N′-bis(ethylene)phosphoramidate having Formula I, or a pharmaceutically acceptable salt, isotopic variant or solvate thereof; wherein the AKR1C3 reductase level of the cell is represented by the AKR1C3 protein level or RNA level and is equal to or greater than a predetermined value.

11. The method according to claim 10, wherein the cell is a cancerous cell.

12. The method according to claim 10, wherein the method further comprises a step for measuring the content of AKR1C3 reductase of cell using AKR1C3 antibodies, where the content of AKR1C3 reductase is measured to be equal to or greater than the predetermined value, the compound is contacted with the cell.

13. (canceled)

14. (canceled)

15. A composition, which comprising:
1) the compound 1-(3-(3-N,N-dimethylaminocarbonyl)phenoxyl-4-nitrophenyl)-1-ethyl-N,N′-bis(ethylene)phosphoramidate having Formula I, or a pharmaceutically acceptable salt, isotopic variant or solvate thereof; and
2) at least one other anti-cancer drug.

16. The composition according to claim 15, wherein the compound is (S)-1-(3-(3-N,N-dimethylaminocarbonyl)phenoxyl-4-nitrophenyl)-1-ethyl-N,N′-bis(ethylene)phosphoramidate having Formula I-1, or (R)-1-(3-(3-N,N-dimethylaminocarbonyl)phenoxyl-4-nitrophenyl)-1-ethyl-N,N′-bis(ethylene)phosphoramidate having Formula I-2.

17. The composition according to claim 15, wherein the anti-cancer drug is selected from the group consisting of gemcitabine, 5-flurouracie (5-FU), sunitinib, abiraterone acetate, prednisolone, erlotinib, meturedepa, uredepa, altretamine, imatinib, triethylenemelamine, trimethylmelamine, chlorambucil, chlornaphazine, estramustine, gefitinib, mechlorethamine, mechlorethamine oxide hydrochloride, melphalan, novembichin, phenesterine, prednimustine, trofosfamide, uracil mustard, carmustine, chlorozotocin, fotemustine, nimustine, ranimustine, dacarbazine, mannomustine, mitobronitol, mitolactol, pipobroman, aclacinomycins, actinomycin, anthramycin, azaserine, bleomycin, cactinomycin, carubicin, carzinophilin, chromomycin, dactinomycin, daunorubicin, daunomycin, 6-diazo-5-oxo-1-norleucine, mycophenolic acid, nogalamycin, olivomycin, peplomycin, plicamycin, porfiromycin, puromycin, streptonigrin, streptozocin, tubercidin, ubenimex, zinostatin, zorubicin, denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, tegafur, L-asparaginase, pulmozyme, aceglatone, aldophosphamide glycoside, aminolevulinic acid, amsacrine, bestrabucil, bisantrene, defofamide, demecolcine, diaziquone, elfornithine, elliptinium acetate, etoglucid, flutamide, hydroxyurea, interferon-alpha, interferon-beta, interferon-gamma, interleukin-2, lentinan, mitoguazone, mitoxantrone, mopidamol, nitracrine, pentostatin, phenamet, pirarubicin, podophyllinic acid, 2-ethylhydrazide, procarbazine, razoxane, sizofiran, spirogermanium, paclitaxel, tamoxifen, erlotonib, teniposide, tenuazonic acid, triaziquone, 2,2′,2″-trichlorotriethylamine, urethan, vinblastine, and vincristine.

18. The composition according to claim 17, wherein the anti-cancer drug is selected from the group consisting of gemcitabine, abiraterone acetate, prednisolone, 5-FU, sunitinib, or the combination of abiraterone acetate and prednisolone.

19. The composition according to claim 18, wherein in the case where the cancer is renal cell carcinoma (RCC), the anti-cancer drug is selected from the group consisting of gemcitabine and sunitinib; in the case where the cancer is gastric cancer, the anti-cancer drug is 5-FU; in the case where the cancer is castrate-resistant prostate cancer (CRPC), the anti-cancer drug is selected from the group consisting of abiraterone acetate and prednisolone or their combination.

20. The composition according to claim 15, wherein the composition further comprises a pharmaceutically acceptable excipient.

21. (canceled)

22. (canceled)

23. (canceled)

24. (canceled)

25. (canceled)

26. (canceled)

27. (canceled)

28. (canceled)

29. (canceled)
